(19) United States
(12) Patent Application Publication
(10) Pub. No.: US 2019/0307594 A1
(43) Pub. Date: Oct. 10, 2019

(54) ORTHOPEDIC SUPPORTS AND METHOD FOR USING THE SAME

(71) Applicant: CSTECH US, Oceanside, CA (US)

(72) Inventors: Chad GIBSON, Carlsbad, CA (US); Jen BARRELLI, Oceanside, CA (US)

(73) Assignee: CSTECH US, Oceanside, CA (US)

(21) Appl. No.: 16/366,801

(22) Filed: Mar. 27, 2019

Related U.S. Application Data
(60) Provisional application No. 62/655,533, filed on Apr. 10, 2018.

(51) Int. Cl. A61F 5/01; A61F 7/02

(52) U.S. Cl. CPC A61F 5/0104; A61F 2007/023; A61F 2007/0219; A61F 7/02

(57) ABSTRACT

Devices and methods for providing orthopedic support to animals. One embodiment is directed to a device for providing orthopedic support to an animal's hock. Another embodiment is directed to a device for providing orthopedic support to an animal's carpal. Another embodiment is directed to a device for providing orthopedic support to an animal's elbow.

### CROSS REFERENCE TO RELATED APPLICATIONS

This application claims the benefit of U.S. Provisional Application No. 62/655,533, filed on Apr. 10, 2018 and entitled “Orthopedic Supports and Method for Using the Same.” The entire contents of the related application are incorporated by reference.

### FIELD

The invention is related to the field of orthopedic supports, and more particularly, to orthopedic supports for animals.

### BACKGROUND

Orthopedic supports are used to stabilize or immobilize a joint of a wearer, most often to reduce motion of the joint while it heals from an injury or other trauma. Historically, types of orthopedic supports include casts, splints, and the Ilizarov apparatus.

Commonly, external fixtures like those described above function by almost completely immobilizing the joint in question. While this may be acceptable for human patients, this proves to be a challenge for animal patients, who more often than not cannot understand the point of immobilizing the supported joint and attempt to move it anyway. It would therefore be advantageous to provide an orthopedic support that imparts increased comfort and/or support for the wearer without sacrificing mobility.

### BRIEF SUMMARY OF THE EMBODIMENTS

Disclosed and claimed herein are devices and methods for providing orthopedic support to animals. One embodiment is directed to a device for providing orthopedic support to an animal's hock. Another embodiment is directed to a device for providing orthopedic support to an animal's carpal. Another embodiment is directed to a device for providing orthopedic support to an animal's elbow.

In one embodiment, the device is configured to treat subacute and chronic conditions of a hock of an animal. In one embodiment, this device comprises a substantially segmented body comprising at least a first and second sheet, both having a side and an opposite side, respectively, the at least a first and second sheets affixed together extending along a first length axis and a second width axis; and a plurality of straps extending along the second axis, connected to at least one of the first and second sheet, connected to substantially opposite side when the wrap is in a wrapped position.

In one embodiment, the device is configured to treat subacute and chronic conditions of a carpal of an animal. In one embodiment, this device comprises a single sheet having a side and an opposite side with a first length axis and a second width axis; and a plurality of straps extending along the second axis, connected to the sheet, connected to substantially opposite side when the wrap is in a wrapped position.

In one embodiment, the device is configured to treat subacute and chronic conditions of an elbow of an animal. In one embodiment, this device comprises a single sheet assembled with a seam down the length of the device so as to form a sleeve and a partial seam across the width of the device to create a bend in the device's shape. In one embodiment, this device also includes a plurality of straps extending along the two ends of the device's sleeve, connected to either side of the bend, designed to wrap around the circumference of the sleeve and fasten the sleeve.

In one embodiment, the device includes interconnected stays that may be easily manufactured and used for various orthopedic applications. In one embodiment, the device includes a plurality of stays secured to the sheets.

In one embodiment, the device includes heating gel affixed to the inside of one or more sheets. In one embodiment, the heating gel is placed in a pocket of the device. In one embodiment, the heating gel is directed towards relaxing muscles around the affected area of the patient.

In one embodiment, the device includes cooling gel affixed to the inside of one or more sheets. In one embodiment, the cooling gel is placed in a pocket of the device. In one embodiment, the cooling gel is directed towards reducing inflammation around the affected area of the patient.

In one embodiment, the plurality of straps includes a means of adjustment. In one embodiment, the plurality of straps is made of either hook or loop fastener strips and the means of adjustment is the fastening of hook and loop fastener strips in different positions. In one embodiment, the hook and loop fastener strip pairs are each a single strip designed to pass through a slot and fasten onto itself.

In one embodiment, the sheets can be made of flexible material configured to extend about and conform to a portion of the patient's anatomy. In one embodiment, the flexible material is flexible in one direction. In one embodiment, the material is flexible in two directions. In one embodiment, the sheets can be partially made of this flexible material.

Other aspects, features, and techniques will be apparent to one skilled in the relevant art in view of the following detailed description of the embodiments.

### BRIEF DESCRIPTION OF THE DRAWINGS

The features, objects, and advantages of the present disclosure will become more apparent from the detailed description set forth below when taken in conjunction with the drawings in which like reference characters identify correspondingly throughout and wherein:

FIG. 1 depicts one embodiment of a device for providing orthopedic support to an animal's hock;

FIG. 2 depicts one embodiment of a device for providing orthopedic support to an animal's carpal; and

FIGS. 3A & 3B depict one embodiment of a device for providing orthopedic support to an animal's elbow.

### DETAILED DESCRIPTION OF THE EXEMPLARY EMBODIMENTS

### Overview and Terminology

One aspect of the disclosure is directed to devices and methods for providing orthopedic support to animals. Embodiments are directed to devices and methods for providing orthopedic support to animals. However, it should be appreciated that the device configurations and methods discussed herein may be applied to other medical purposes.

References throughout this document to “one embodiment,” “certain embodiments,” “an embodiment,” or similar terms mean that a particular feature, structure, or characteristic described in connection with the embodiment is included in at least one embodiment. Thus, the appearances of such phrases in various places throughout this specification are not necessarily all referring to the same embodiment. Furthermore, the particular features, structures, or characteristics may be combined in any suitable manner on one or more embodiments without limitation.

### Exemplary Embodiments

In one embodiment, the device disclosed herein is directed to providing orthopedic support to a body part for an animal, including humans. In one embodiment, the body part is a joint. Non-limiting examples of joints include knees, elbows, hock, carpal, fetlock, stifle, and the like.

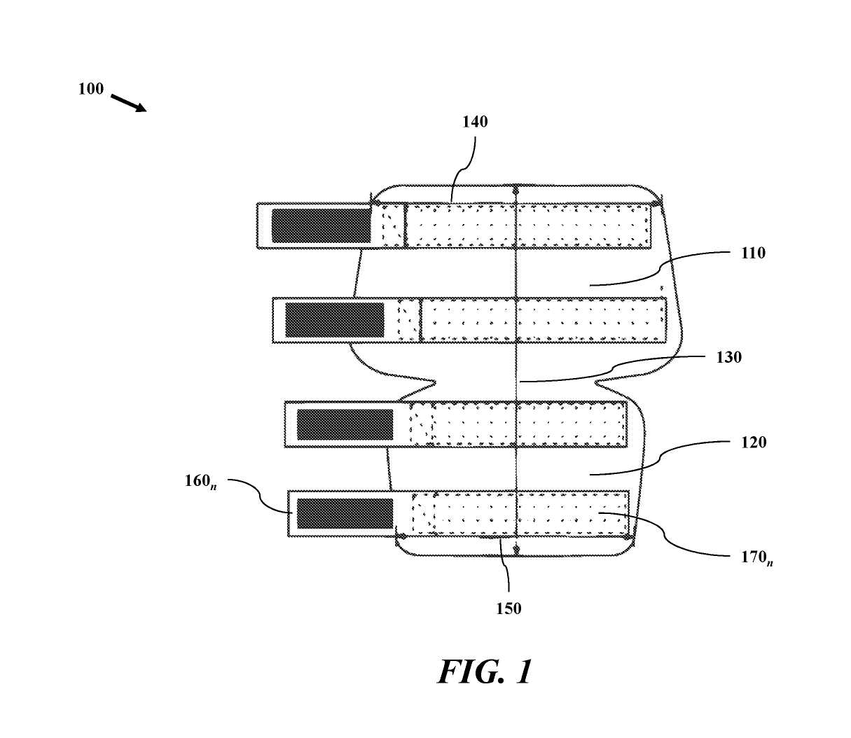

FIG. 1 depicts one embodiment of a device for providing orthopedic support to an animal's hock. Device 100 comprises a first sheet 110 and a second sheet 120 joined together at a single edge. Length axis 130 describes the total length of the two connected sheets. First width axis 140 and second width axis 150 describe the widths of their respective sheets. The sheets are designed such that first width axis 140 is wide enough for first sheet 110 to wrap completely around the portion of a patient's limb above the hock and second width axis 150 is wide enough for second sheet 120 to wrap completely around the portion of a patient's limb below the hock.

In one embodiment, first sheet 110 and/or second sheet 120 are single layer. In one embodiment, first sheet 110 and/or second sheet 120 have at least two layers.

In one embodiment, device 100 includes heating gel affixed to the inside of first sheet 110, second sheet 120, or both. In one embodiment, the heating gel is inserted between layers of the multilayer first sheet 110 and/or multilayer second sheet 120. In one embodiment, the heating gel is placed in a pocket of device 100. In one embodiment, the heating gel is directed towards relaxing muscles around the affected area of the patient.

In one embodiment, device 100 includes cooling gel affixed to the inside of first sheet 110, second sheet 120, or both. In one embodiment, the cooling gel is inserted between layers of the multilayer first sheet 110 and/or multilayer second sheet 120. In one embodiment, the cooling gel is placed in a pocket of device 100. In one embodiment, the cooling gel is directed towards reducing inflammation around the affected area of the patient.

In one embodiment, first sheet 110, second sheet 120, or both can be made of flexible material configured to extend about and conform to a portion of the patient's anatomy. In one embodiment, the sheets can be partially made of this flexible material. In one embodiment, the material is flexible in one direction. In one embodiment, the material is flexible in two directions.

In one embodiment, a seam joins first sheet 110 and second sheet 120. In one embodiment, that seam is flexible. In another embodiment, first sheet 110 and second sheet 120 are manufactured out of a single piece and no joining is required. In this embodiment, the two halves of the single piece should still be considered as first sheet 110 and second sheet 120.

In one embodiment, device 100 includes interconnected stays that may be easily manufactured and used for various orthopedic applications. In one embodiment, device 100 includes a plurality of stays secured to first sheet 110, second sheet 120, or both.

In one embodiment, each sheet has at least one fastening mechanism. Non-limiting fastening mechanisms include hook and loop, hook and eye, elastic bands, clips, buckles, snaps, buttons, eyelets, and the like. In one embodiment, each sheet has a plurality of hook fastener strips 160₁₋ₙ and loop fastener strips 170₁₋ₙ. Hook fastener strips 1601, extend beyond the length of first width axis 140 or second width axis 150, while loop fastener strips 170₁₋ₙ run along the width of their respective sheets. In one embodiment, loop fastener strips 170₁₋ₙ cover a portion of their respective sheets that is a shorter distance than the length of first width axis 140 or second width axis 150. In one embodiment, each hook fastener strip 160ₓ runs along the same axis as a loop fastener strip 170ₓ and in one embodiment is connected to that loop fastener strip 170ₓ. In one embodiment, each hook fastener strip 160ₓ may fasten to its corresponding loop fastener strip 170ₓ to secure their respective sheets around the circumference of the patient's limb once wrapped. The hook fastener strip 160ₓ may fasten to its corresponding loop fastener strip 170ₓ at different locations in order to provide an adjustable circumference around the patient's limb. In one embodiment, each hook fastener strip 160,, first passes through a slot before fastening to its corresponding loop fastener strip 170ₓ.

In one embodiment, device 100 lacks a complete complement of loop fastener strips 170₁₋ₙ and instead has the first sheet 110, second sheet 120, or both made partially or entirely of a material to which hook fastener strips 160₁₋ₙ may fasten.

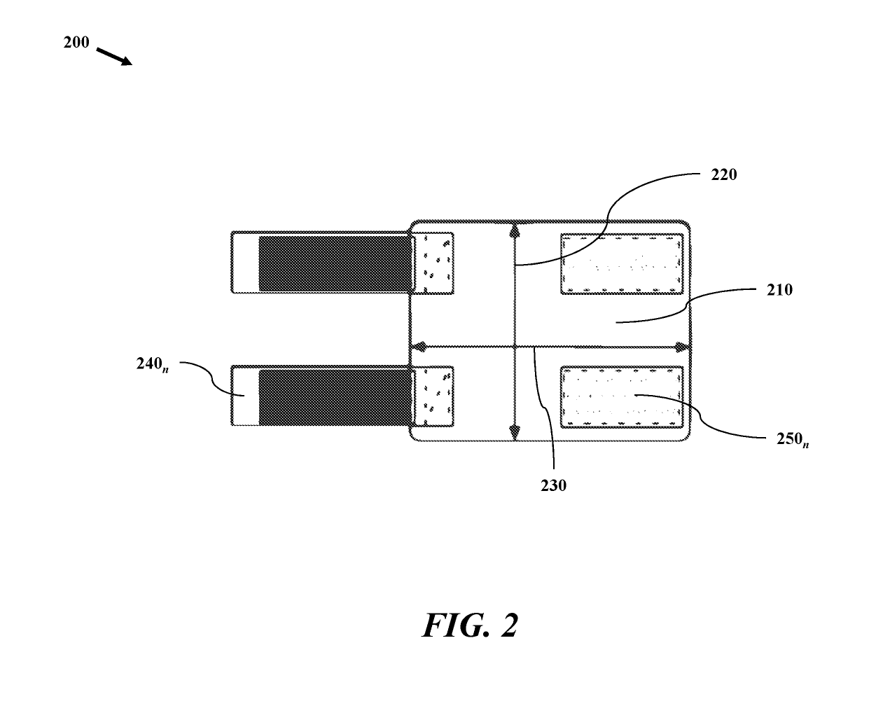

FIG. 2 depicts one embodiment of a device for providing orthopedic support to an animal's carpal. Device 200 comprises a sheet 210. Length axis 220 describes the length of the sheet. Width axis 230 describes the width of the sheet. The sheet is designed such that width axis 230 is wide enough for sheet 210 to wrap completely around the patient's carpal.

In one embodiment, sheet 210 is a single layer. In one embodiment, sheet 210 has at least two layers.

In one embodiment, device 200 includes heating gel affixed to the inside of sheet 210. In one embodiment, the heating gel is inserted between layers of the multilayer first sheet 210. In one embodiment, the heating gel is placed in a pocket of device 200. In one embodiment, the heating gel is directed towards relaxing muscles around the affected area of the patient.

In one embodiment, device 200 includes cooling gel affixed to the inside of sheet 210. In one embodiment, the cooling gel is inserted between layers of the multilayer sheet 210. In one embodiment, the cooling gel is placed in a pocket of device 200. In one embodiment, the cooling gel is directed towards reducing inflammation around the affected area of the patient.

In one embodiment, sheet 220 can be made of flexible material configured to extend about and conform to a portion of the patient's anatomy. In one embodiment, the sheets can be partially made of this flexible material. In one embodiment, the material is flexible in one direction. In one embodiment, the material is flexible in two directions.

In one embodiment, device 200 includes interconnected stays that may be easily manufactured and used for various orthopedic applications. In one embodiment, device 200 includes a plurality of stays secured to sheet 210.

In one embodiment, sheet 210 has at least one fastening mechanism. Non-limiting fastening mechanisms include hook and loop, hook and eye, elastic bands, clips, buckles, snaps, buttons, eyelets, and the like. In one embodiment, the sheet has a plurality of hook fastener strips 240₁₋ₙ and loop fastener strips 250₁₋ₙ. Hook fastener strips 240₁₋ₙ extend beyond the length of width axis 230, while loop fastener strips 250₁₋ₙ run along the width of the sheet. In one embodiment, loop fastener strips 240₁₋ₙ cover a portion of the sheet that is a shorter distance than the length of width axis 230. In one embodiment, each hook fastener strip 240ₓ runs along the same axis as a loop fastener strip 250₁₋ₙ and in one embodiment is connected to that loop fastener strip 250ₓ. In one embodiment, each hook fastener strip 240ₓ may fasten to its corresponding loop fastener strip 250ₓ to secure their respective sheets around the circumference of the patient's limb once wrapped. The hook fastener strip 240ₓ may fasten to its corresponding loop fastener strip 250ₓ at different locations in order to provide an adjustable circumference around the patient's limb. In one embodiment, each hook fastener strip 240ₓ first passes through a slot before fastening to its corresponding loop fastener strip 250ₓ.

In one embodiment, device 200 lacks a complete complement of loop fastener strips 250₁₋ₙ and instead has the sheet 210 made partially or entirely of a material to which hook fastener strips 240₁₋ₙ may fasten.

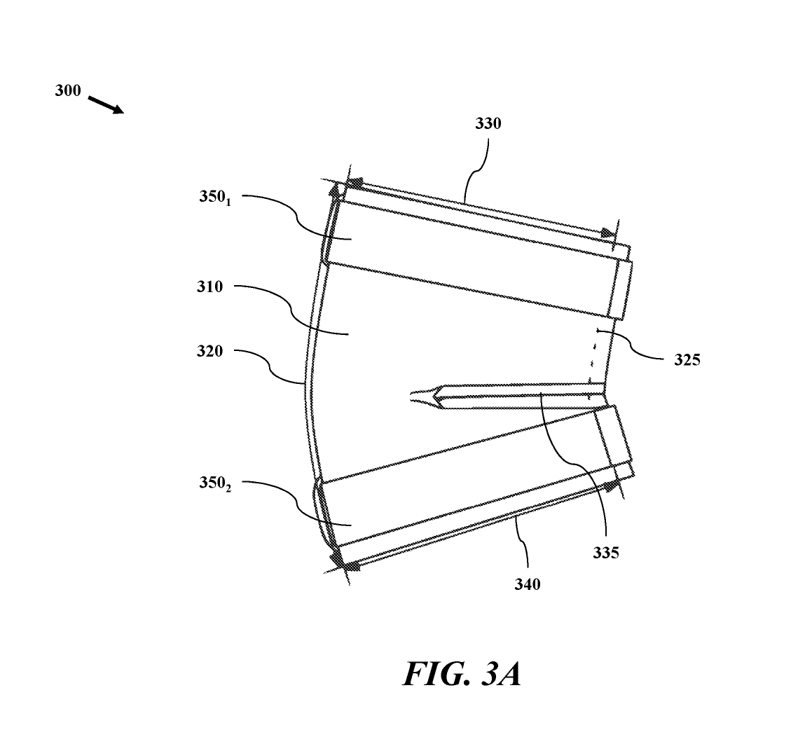

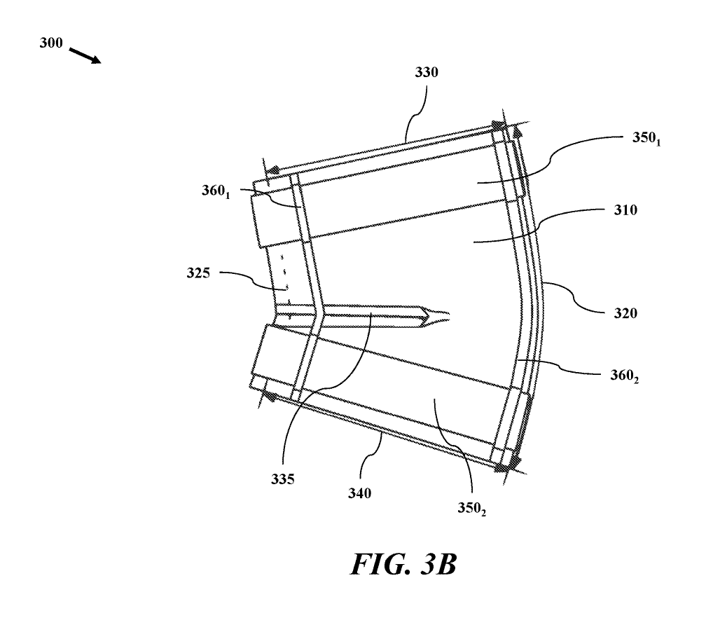

FIGS. 3A & 3B depict one embodiment of a device for providing orthopedic support to an animal's elbow. Device 300 comprises sheet 310 assembled with a seam 325 down the length of the device so as to form a sleeve and a partial seam 335 across the width of the device to create a bend in the device's shape. Length axis 320 describes the length of the sheet along its longest point. First width axis 330 and second width axis 340 describe the width of each end of the sleeve between the fold along length axis 320 and seam 325. In one embodiment, first width axis 330 is the same length as second width axis 340. In one embodiment, first width axis 330 is longer or shorter than second width axis 340.

In one embodiment, sheet 310 is single layer. In one embodiment, sheet 310 has at least two layers.

In one embodiment, device 300 includes heating gel affixed to the inside of sheet 310. In one embodiment, the heating gel is inserted between layers of the multilayer sheet 310. In one embodiment, the heating gel is placed in a pocket of device 300. In one embodiment, the heating gel is directed towards relaxing muscles around the affected area of the patient.

In one embodiment, device 300 includes cooling gel affixed to the inside of sheet 310. In one embodiment, the cooling gel is inserted between layers of the multilayer sheet 310. In one embodiment, the cooling gel is placed in a pocket of device 300. In one embodiment, the cooling gel is directed towards reducing inflammation around the affected area of the patient.

In one embodiment, sheet 320 can be made of flexible material configured to extend about and conform to a portion of the patient's anatomy. In one embodiment, the sheets can be partially made of this flexible material. In one embodiment, the material is flexible in one direction. In one embodiment, the material is flexible in two directions.

In one embodiment, device 300 includes interconnected stays that may be easily manufactured and used for various orthopedic applications. In one embodiment, device 300 includes a plurality of stays secured to sheet 310.

In one embodiment, sheet 310 has at least one fastening mechanism. Non-limiting fastening mechanisms include hook and loop, hook and eye, elastic bands, clips, buckles, snaps, buttons, eyelets, and the like. In one embodiment, each end of the sleeve has a hook/loop fastener strip 350ₓ. Hook/loop fastener strips 350₁ and 350₂ extend beyond the length of their respective width axes 330 and 340 and connect to the sleeve at a limited surface portion of the sleeve. In one embodiment, the hook portion of each hook/loop fastener strip 350ₓ covers part or all of the area where the hook/loop fastener strip 350ₓ joins with the sleeve. In one embodiment, the portion of each hook/loop fastener strip 350ₓ that is not joined with the sleeve extends long enough to wrap around the total circumference of its respective sleeve end.

In one embodiment, device 300 includes one or more stays 360ₓ running along the length of the sleeve at different sections. In one embodiment, each stay 360ₓ is configured to include a loop through which the patient may thread each hook/loop fastener strip 350ₓ.

In one embodiment, sheet 310 is made partially or entirely of a material to which a hook fastener strip 350ₓ may fasten. In this embodiment, each hook/loop fastener strip 350ₓ is instead a hook fastener strip.

Unless defined otherwise, all technical and mechanical terms used herein have the same meaning as is commonly understood by one of ordinary skill in the art to which this disclosure belongs. All patents, applications, published applications and other publications referred to herein are incorporated by reference in their entirety. If a definition set forth in this section is contrary to or otherwise inconsistent with a definition set forth in applications, published applications and other publications that are herein incorporated by reference, the definition set forth in this document prevails over the definition that is incorporated herein by reference.

While various embodiments of the present disclosure have been described above, it should be understood that they have been presented by way of example only, and not of limitation. Likewise, the various diagrams may depict an example architectural or other configuration for the invention, which is done to aid in understanding the features and functionality that can be included in the invention. The invention is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present invention. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the invention may be described or claimed in the singular, the plural is contemplated to be within the scope thereof unless limitation to the singular is explicitly stated.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed across multiple locations.

What is claimed is:

1. A device for providing orthopedic support for a user comprising a sheet and at least one fastener;
wherein the sheet comprises a first surface, a second surface, a top edge, a bottom edge, a right side, and a left side;
wherein the sheet wraps around a body part of the user with the second surface of the sheet contacting the body part;
wherein the at least one fastener is attached to the first surface of the sheet
wherein the at least one fastener attaches the right side to the left side to hold the device in place.

2. The device of claim 1, wherein the sheet is made from a flexible material.

3. The device of claim 1, wherein the sheet has at least two layers.

4. The device of claim 1, wherein the at least one fastener is a hook and loop type of fastener.

5. The device of claim 1, further comprising a heating or cooling composition.

6. The device of claim 1, further comprising at least one support stay.

7. The device of claim 1, wherein the sheet is shaped to fit around a hock of an animal.

8. The device of claim 1, wherein the sheet is shaped to fit around a carpal of an animal.

9. The device of claim 1, wherein the sheet is shaped to fit around an elbow of an animal.

10. A method of treating subacute or chronic conditions in an animal, the method comprising the steps of wrapping the device of claim 1 around an affected body part of the animal and fastening the at least one fastener.
